(19) United States
(12) Patent Application Publication
(10) Pub. No.: US 2014/0031573 A1
(43) Pub. Date: Jan. 30, 2014

(54) Enhancing Oil Accumulation in Vegetative Tissue of Plants

(71) Applicant: Brookhaven Science Associates, LLC, Upton, NY (US)

(72) Inventors: John Shanklin, Shoreham, NY (US); Hiu Liu, Ridge, NY (US); Changcheng Xu, Calverton, NY (US); Jilian Fan, Calverton, NY (US); Xuebin Zhang, Ridge, NY (US)

(73) Assignee: Brookhaven Science Associates, LLC, Upton, NY (US)

(21) Appl. No.: 13/933,196

(22) Filed: Jul. 2, 2013

Related U.S. Application Data
(60) Provisional application No. 61/667,077, filed on Jul. 2, 2012.

(51) Int. Cl. C12N 15/82

(52) U.S. Cl. CPC C12N 15/8247
USPC 554/227; 435/320.1; 800/298; 800/264

(57) ABSTRACT

Combinations of genes are used to enhance the accumulation of triacylglycerol compounds in vegetative tissues of plants. Fatty acids in the form of triacylglycerol compounds accumulate in vegetative tissues in excess amounts compared to untreated plants.

This application claims benefit of U.S. Provisional Application No. 61/667,077 filed Jul. 2, 2012, the contents of which are incorporated herein by reference.

### GOVERNMENT SUPPORT

This invention was made with Government support under contract number DE-AC02-98CH10886, awarded by the U.S. Department of Energy and the U.S. Department of Energy ARPA-E grant DE-AR00000470. The Government has certain rights in the invention.

### BACKGROUND

Triacylglycerol compounds (TAGs), which consist of three fatty acids (FA) esterified to a glycerol backbone, are the predominant store of carbon in the majority of seeds and particularly seeds of oilseed crops. While they may account for more than 60% of the weight of seeds, triacylglycerols normally accumulate only to very low levels in non-seed/vegetative tissues, typically <0.1% (Yang and Ohlrogge, 2009, Plant Physiol. 150:1981-1989). Fatty acids from the seeds of oilseed crops have long been recognized for their nutritional value and for their use as industrial feedstocks. More recently their use as biodiesel is being contemplated and encouraged. However, using today's commercial oilseed crops to meet biodiesel production targets is not currently feasible.

Because the biomass ratio of vegetative tissue to seed tissue in crop and non-crop plants is so substantial, it is an intuitive certainty that a small, but significant, increase in the weight percent of TAGs stored in non-seed biomass could provide an enormous boost to the amount of biodiesel that could be produced during a single growth cycle. To accomplish this one needs to first understand and then to manipulate the metabolic controls that normally limit the accumulation of fatty acids, particularly in the form of TAGs, in the vegetative tissues of plants. Various preliminary studies have suggested different lines of approach.

TAGs have been shown to accumulate in non-seed tissues in a variety of plant mutants. For example, mutation of the Arabidopsis homologue of the human CGI-58 gene (At4G24160) resulted in the accumulation of neutral lipid droplets in mature leaves (James, et al. 2010, Proc. Natl. Acad. Sci. USA 107:17833-17838 and US20100221400A1). Mutation of trigalactosyldiacylglycerol (TGD) proteins (tgd1 mutants) involved in fatty acid (FA) transport from the endoplasmic reticulum (ER) to the plastid had a similar effect (Xu, et al. 2010 Plant Cell Physiol. 2010, 51:1019-1028). Mutations of pickle, a chromatin remodeling factor involved in switching from embryonic expression to vegetative expression, lead to TAG accumulation in vegetative tissue (Ogas, et al. 1999, Proc. Natl. Acad. Sci. USA 96:13839-13844). However none of these mutant plant strains accumulated TAG levels in excess of about one percent of dry weight.

The accumulation of biological molecules is a balance between the rate of synthesis and the rate of degradation. Disruption of fatty acid breakdown, which occurs via β-oxidation in the peroxisome, can lead to increased TAG levels. The pathway is initiated by uptake of FA via a peroxisomal ABC transporter, CTS, followed by β-oxidation within the organelle. Slocombe et al. (Plant Biotech. J. (2009) 7:694-703) show that leaf TAG levels can be increased significantly (10-20 fold) by blocking fatty acid breakdown. They surmised that their results suggest that recycled membrane fatty acids can be captured as TAG compounds by expressing seed program genes in senescing tissue or by blocking fatty acid breakdown, or both. Together, data from the tgd1 mutants and the CTS mutants suggest that the increased TAG accumulation is a response to increased FA supply levels.

A transcription factor, wrinkled1 (WRI1) (Cernac and Benning (2004) The Plant J. 40:575-585) controls the coordinate expression of many genes of fatty acid synthesis (FAS) and therefore represents an excellent target for increasing the supply of fatty acids (Pouvreau et al. 2011, Plant Physiol. 156:674-686). Seedlings expressing WRI1 require elevated glucose or fructose levels in order to facilitate increases in vegetative TAG (Cernac and Benning, 2004).

The starch biosynthetic pathway is a competing sink for photosynthetic carbon (for example, see Fan et al. 2012, Plant Cell Physiol. 53:1380-1390). Down-regulation of this pathway using an RNAi approach targeting ADP-glucose pyrophosphorylase with AGPRNAi in WRI1 overexpressing lines increased the levels of hexoses and the level of TAG accumulation was 6-fold higher than in lines overexpressing either mutant alone (Sanjaya et al. 2011, Plant Biotechnology J. 9:874-853).

In another promising approach, a second transcription factor leafy cotyledon 2 (LEC2) (Santos Mendoza et al. 2005, FEBS Lett. 579:4666-4670), which is epistatic to WRI1, was co-expressed in the cts-2 mutant, resulting in oil accumulation in vegetative tissue (Slocombe et al., 2009). While this approach yielded interesting results, the expression of LEC2 can have undesirable pleiotropic effects.

None of these early studies has established commercially relevant plant strains having a small, but significant, increase in the weight percent of TAGs stored in vegetative tissue (non-seed biomass).

### SUMMARY

A combination of genes is described for enhancing the accumulation of triacylglycerol fatty acids in vegetative tissues of plants when introduced for expression in such plants. Also described herein is a method for enhancing the accumulation. This involves introducing into the plant the combination of genes for expression. The combination of genes may include for example, genes that generally increase fatty acid synthesis, genes that encode oleosin or other similar proteins, genes that encode diacylglycerol acyltransferases, genes that encode phospholipid:diacylglycerol acyltransferases, genes that encode medium chain thioesterases and combinations of such genes. As a result of the present combination of genes and methods for using an altered crop plant may be generated. The altered crop plant may have a weight percent of triacylglycerol compounds (TAGs) in one or more vegetative tissues that is at least two-fold higher than the weight percent in one or more vegetative tissues of a parent plant from which the altered crop plant was derived. Also, described is an altered crop plant wherein a harvestable TAG is twenty-fold higher than a harvestable TAG from one or more vegetative tissues of its parent plant.

### DRAWINGS

FIG. 1: Depiction of normal leaf metabolism showing the assembly of TAG in the cytoplasm and/or endoplasmic reticulum (ER).

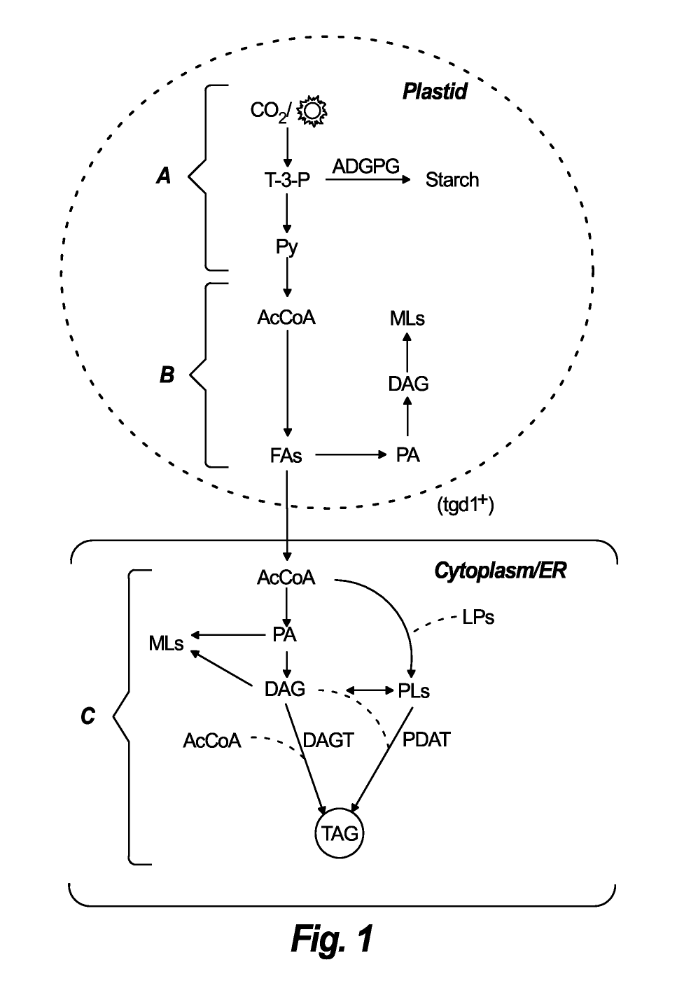

FIG. 2: Depiction of modified leaf metabolism showing the proposed effect of co-expression of four genes, wrinkled1 (WRI1); medium chain thioesterase (MCT or T); diacylglycerol acyltransferase (DGAT) and olesin 1 (Ole1).

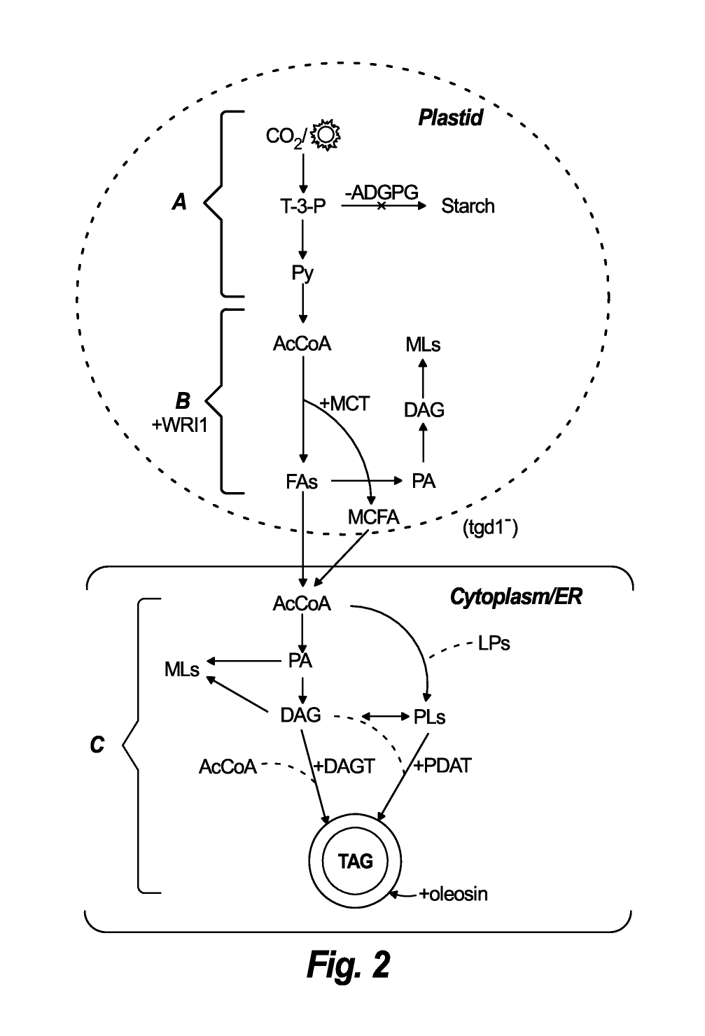

FIG. 3: Thin layer chromatography showing results of transient expression in Nicotiana benthamiana leaves by the control inoculant (C); Ole1 inoculant (O); DGAT inoculant (D); WRI1 inoculant (W); Ole1, DGAT, and WRI1 triple inoculant (ODW) and Oli1, DGAT, WRI1 and MCT tetra inoculant (ODWT). Gene Bank accession numbers for the genes used in initial experimentation are shown and their references include DGAT1, Xu et al., 2008 Plant Biotechnology Journal. 6:799-818; OLE1, Shimada and Hara-Nishimura 2010 Biol. Pharm. Bull. 33:360-363; OLE1, Hu et. al. 2009 Plant Cell Rep 28:541-549; WRI1, Cernac A, Benning C. 2004 Plant J. 40:575-85; Vegetative TAG, Sanjaya et. al., 2011 Plant Biotechnology Journal, 9:874-883; MCT Voelker et. al., 1992, Science 257:72-74.

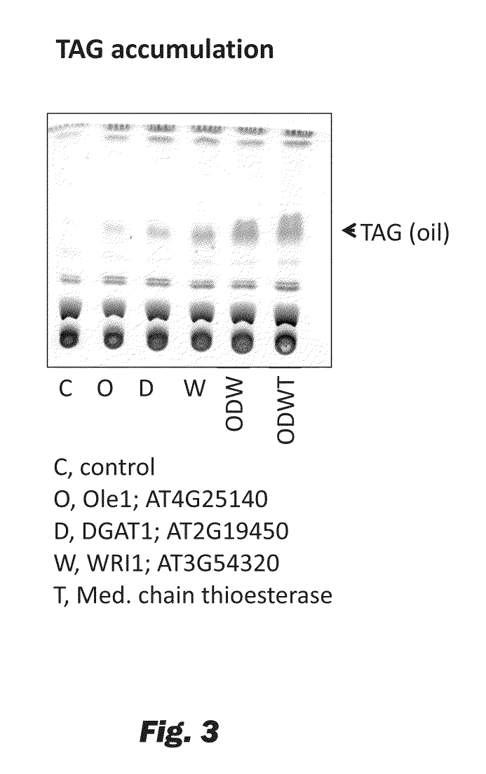

### DETAILED DESCRIPTION

High oil content in vegetative tissues of plants may be achieved using a balance of up-regulation and down-regulation of various interacting and competing metabolic pathways. Fatty acid synthesis and accumulation in general may be up-regulated through a combination of a strategic choice of the genetic background of the plant and, potentially, overexpressing fatty acid synthase genes and suppressing genes diverting fatty acids to other pathways. The transfer of fatty acids from various metabolic precursors to form triacylglycerols may be enhanced, nascent oil bodies may be protected, for example, by coating with protein, and FA turnover or diversion to other carbon sink pathways may be suppressed.

Up-regulation of FAS is achieved by ectopic expression of Arabidopsis WRI1 (Pouvreau et al. 2011). Expression of higher level transcription control factors such as LEC2 and or FUS3 (the B-3 domain transcription factor FUSCA3) may be considered while recognizing that this may increase the possibility that pleiotropic effects could be substantial.

Expression of DGAT along with WRI1 enhances increases in TAG accumulation, indicating that its levels are limiting the conversion of FA-CoAs to TAG via the Kennedy pathway (Xu et al. 2008, Plant Biotechnol. 6:799-818; Zheng et al. 2008, Nat. Genetics 40:367-372).

Because of the complementary overlapping function of phospholipid:diacylglycerol acyltransferase 1 (PDAT1; At5g13640) and acyl-CoA:diacylglycerol acyltransferase 1 (DGAT1) (Zhang et al. 2009, The Plant Cell 18:3885-3901), overexpression of PDAT1 also enhances TAG accumulation. Overexpression of PDAT1 in a mutant background in which sub-cellular lipid transfer between the endoplasmic reticulum (ER) and thylakoid is disrupted (the tdg1 Arabidopsis mutant, see Xu, et al. 2003) leads to considerable enhancement of TAG accumulation in vegetative tissue.

Creating a stable storage pool for vegetative TAG accumulation by ectopically expressing oleosins, key proteins that coat oil bodies in seeds, is beneficial to this effort because oleosin expression levels are normally very low in vegetative tissues (Shimada and Hara-Nishimura 2010, Biol. Pharm. Bull. 33:360-363). In Brassica napus the level of oleosin expression is correlated with oil content (Hu et al. 2009, Plant Cell Rep. 28:541-549), and in Arabidopsis mutants deficient in oleosins, oil content is decreased relative to wild type (Siloto et al. 2006, Plant Cell 18: 1961-1974). Further, the ectopic expression of oleosin in non-seed tissues of Arabidopsis led to FA accumulation in the ER, suggesting that such a background would be ideal for oil body formation and chaperoning newly synthesized FA from the ER into oil bodies (Beaudoin and Napier 2000, Planta 210:439-445). Co-expression of Arabidopsis OLE1, the major seed isoform, along with WRI1 and DGAT1 and co-expression of OLE1 and PDAT1 in the tgd1 strain both lead to substantial accumulation of TAG in vegetative tissue. Co-expression of WR1, DGAT1, PDAT1 and OLE1 in a tgd1 strain may produce additional accumulation of TAGs. Combining enhanced expression of these genes with down-regulation of competing pathways, the starch synthetic pathway and the β-oxidation breakdown pathway, is also contemplated to enhance TAG accumulation in vegetative tissues.

Suppression/down-regulation of competing pathways for carbon storage, in particular the starch synthesis pathway, is complementary to the up-regulation improvements in TAG accumulation in vegetative tissue. Using RNAi to down-regulate AGP, the gene encoding the key enzyme ADP-Glucose pyrophosphorylase reduces the diversion of triose phosphates into the competing, starch biosynthetic pathway.

In addition to down-regulating the strength of a competing carbon sink, providing high levels of sugar may lead to increased accumulation of TAG as was shown for expression of WRI1 in high sugar lines of tobacco (Andrianov et al. 2010, Plant Biotechnol. J. 8:277-287). p024 Another consideration to improving TAG accumulation is to prevent the β-oxidation degradation of the FAs that accumulate in vegetative tissues (see Kunz, et al. 2009, The Plant Cell 21:2733-2749). To achieve down-regulation of a-oxidation the ABC transporter, PXA1, and the core β-oxidation enzyme, KAT2, may be suppressed, knocked out or knocked down by appropriate means.

Another gene considered as a target for suppression is the CTS-2 (COMATOSE locus), which appears to regulate transport of Acyl-CoAs to the peroxisome (Foottit et al. 2002, EMBO J. 21:2912-2922). As in the case of over-expression of the higher level transcription control elements, suppression of CTS-2 may lead to additional pleiotropic effects.

Expression of a medium chain thioesterase (MCT) in Brassica napus resulted in the accumulation 60 mol % of laurate in TAG (Voelker et al. 1992, Science 257:72-74). Further investigation revealed that only 50% of the laurate synthesized was recovered in lipid, and that enzymes of β-oxidation were elevated in these plants suggesting that both FAS and β-oxidation were induced upon the expression of MCT. (Eccleston and Ohlrogge, 1998, Plant. Cell 10:613-621). MCT interrupts FAS releasing C12 FA from 12:0-ACP, thereby decreasing the levels of long chain acyl-ACPs. Increased FAS is attributable to increased acetyl-CoA carboxylase (ACCase) activity upon the removal of product inhibition by long chain acyl-ACPs (Shintani and Ohlrogge, 1995, Plant Journal 7:577-587); the feedback signal is now identified as 18:1-ACP (Andre, et al. 2012, Proc. Natl. Acad. Sci. USA 109:10107-10112). Thus, a present strategy involves introduction of the MCT to reduce ACCase inhibition by terminating the ACP track of FA biosynthesis at the C12 step, thus lowering the accumulation of the inhibitory signal, 18:1-ACP. MCT may be co-expressed with OLE1, WRI1 and DGAT1, and possibly PDAT1. These constructs may be assessed for their ability to convey increased TAG accumulation. Subsequently an attempt may be made to reduce β-oxidation by down-regulation of PXA1 or CTS-2 and KAT2 with the use of appropriate RNAi constructs. Using the tgd1 background strain with these combinations is also contemplated.

Relatively rapid testing of various co-expression combinations can be achieved using the Nicotiana benthamiana transient expression system as described by Petri et al. (Plant Methods (2010) 6:8), with minor modifications. Genes may be expressed under the control of the 35S promoter. To test combinations of genes, single genes are established in individual Agrobacterium lines. These are combined and co-infiltrated into the abaxial surface of the tobacco leaves. All co-infections are performed with the P19 protein. After phenotypic screening for TAG content, thin layer chromatograph and ESI mass spectrometry (Bates, et al. 2009, Plant Physiol. 150:55-72) may be used to quantitate the contribution of each gene to oil (TAG) accumulation.

Despite the fact that the quantitation in the transient expression scheme is better within than between experiments, the rapid studies provide indicators of the optimal combinations to be processed in stable transformation and genetic line development. Optimal combinations of genes identified in the transient expression analyses are used to establish stably transformed Arabidopsis strains, tobacco transformants and additional plants such as sugar cane and sorghum. In Arabidopsis, well characterized knock out and knock down mutants of appropriate nature can be used as the genetic background recipients of the gene combinations.

Over expression of the combination of genes: 1) WRI1, DGAT1 and OLE1; 2) WRI1, DGAT1, OLE1 and MCT; 3) WRI1, DGAT1, PDAT1, and OLE1; and etc., in vegetative tissues of crop plants, or non-crop and specifically energy crop/non-crop (e.g., miscanthus, camelina) plants will serve for purposes of increasing the levels of storage oil (TAG) in such tissues. It is anticipated to combine FUSS and LEC2 as transcription factors that control the expression of WRI1 either in place of WRI1 or in combination with it to achieve further enhancements in TAG accumulation. Tgd1 mutant recipient strains for transformation of the gene combination may be preferred.

Further enhancements of vegetative TAG accumulation are expected by suppressing ADP-G pyrophosphorylase, i.e., the competing starch pathway and by suppressing PXA1, a component of the peroxisomal FA uptake complex.

Generation of stable transgenic plants expressing various combinations of the genes identified herein may result in crop plants capable of generating valuable oil compositions in tissues more typically considered to be sources of ‘bio-ethanol’ or material for re-cycling by plowing into fallow fields (e.g., corn stover and the like). The production of useful oil compositions in vegetative tissues of plants greatly enhances the energy density of plant tissue.

Vegetative plant tissue may be defined as any non-seed plant tissue, examples of which include leaf, stem, root, tuber, bark, and the like. Vegetative plant tissue comprises the non-seed biomass of plants. For purposes of harvesting TAG from plant biomass, seed may be harvested prior to harvesting the remaining plant, but seed may be included in the total harvest of the plant for recovery of TAG.

Incorporation of the genes into expression constructs having appropriate promoters and regulatory sequences may generate plants having coordinated expression of the combinations of genes in tissues of choice, depending upon the targeted crop plant. The choice of promoter and regulatory sequences will be governed by the selection of the target tissue in the target plant.

Preliminary experiments are carried out using constitutive promoters for controlling expression of the introduced activating or inhibitory genes. Constitutive promoters, tissue-specific promoters, growth stage-specific promoters, and inducible promoters may be independently selected for use for each introduced gene and a particular promoter type may be used for enhancing genes and a separate or identical or similar particular type for inhibitory genes that are introduced. Combinations of promoters for the various genes that produce the optimal accumulation of TAG compounds in vegetative tissue with the least disruption of plant growth and seed productivity and germination are preferred.

A small but significant enhancement of the accumulation of TAGs (weight percent) in non-seed/vegetative tissue means an increase in TAG weight percent of greater than 2-fold over the weight percent of TAGs in the same vegetative tissue of the plant from which the altered plant was originally derived (parent plant). Preferred embodiments are plants in which TAG weight percent in vegetative tissue is more than 5-fold higher than the weight percent in the same tissue of the parent plant and even more preferred embodiments are plants in which TAG weight percent is 10-fold and especially preferred embodiments are plants in which TAG weight percent is 20-fold or more increased over the weight percent in the same tissue of the parent plant. An upper limit of the increase in the weight percent of TAG in the vegetative tissue may be the weight percent wherein for a particular plant species plant growth, seed production and/or seed germination are negatively affected to such an extent that there is no net gain in harvestable TAG.

Genes and gene combinations that may be overexpressed include for example, genes that generally enhance fatty acid synthesis, genes encoding diacylglycerol acyl transferases (e.g., DGAT1 and PDAT1), genes encoding oleosin and oleosin-like proteins (proteins that coat or protect oil droplets in cells) (e.g., OLE1) and genes for medium chain thioesterases.

The genetic background of plants to receive these combinations of genes may include for example, tdg1 mutants, and/or mutant strains with knocked out or knocked down genes encoding enzymes of competing carbon pathways, such as the starch synthetic pathway or the fatty acid breakdown (e.g., β-oxidation pathway). The availability and viability of such recipient strain will dictate their selection.

Genes and gene combinations for expression may also include genes encoding, for example, RNAi constructs for inhibiting competing carbon pathways in plant cells. The genes targeted by such RNAi constructs include genes of the starch biosynthetic pathway (e.g., AGD) and genes for the β-oxidation pathway (e.g., PXA1 and KAT2)

Each of the genes to be introduced, whether for enhancement or reduction of activity, will be introduced in an expression configuration that optimizes the desired outcome (a small, but significant increase in TAG accumulation in vegetative tissue) while minimizing potential negative effects of the altered metabolism on plant growth, seed production and seed germination. The optimal expression configuration may include the optimal selection of the promoter from constitutive, inducible, tissue-specific, growth stage-specific and the like.

Plants optimally expressing the enhancement and reduction of activity genes may be developed such that they have suitably normal growth, suitably normal seed production and suitably normal seed germination. Such plants may further carry other transgene conferring traits such as disease resistance and/or herbicide or pesticide resistance.

1. A combination of genes for enhancing accumulation of triacylglycerol fatty acids in vegetative tissues of plants when introduced for expression therein.

2. The combination of genes according to claim 1 wherein the genes are selected from the group consisting of genes which generally increase fatty acid synthesis, genes encoding oleosin or similar proteins, genes encoding acyl CoA:diacylglycerol acyltransferases, genes encoding phospholipid: diacylglycerol acyltransferases, genes encoding medium chain thioesterases and combinations thereof.

3. The combination of genes according to claim 2 further including genes encoding transcription factors.

4. The combination of genes according to claim 2 further including genes for inhibiting competing pathways.

5. The combination of genes according to claim 3 further including genes for inhibiting competing pathways.

6. The combination of genes according to claim 4 or claim 5 wherein the competing pathway leads to starch synthesis.

7. The combination of genes according to claim 6 wherein the gene for inhibiting starch synthesis is an ADP-glucose pyrophosphorylase RNAi gene.

8. The combination of genes according to claim 4 or claim 5 wherein the competing pathway leads to fatty acid degradation.

9. The combination of genes according to claim 8 wherein fatty acid degradation is through β-oxidation.

10. The combination of genes according to claim 9 wherein the gene for inhibiting β-oxidation is selected from the PXA-1 gene and the KAT2 gene or a combination thereof.

11. A plant containing the gene combination of claim 1.

12. The plant according to claim 11 having a tgd1 mutation such that oligogalactolipids accumulate in leaves.

13. The plant of claim 11 wherein TAG accumulation in leaf tissue is enhanced.

14. The plant of claim 11 wherein TAG accumulation in stems is enhanced.

15. The plant of claim 11 wherein TAG accumulation in roots is enhanced.

16. The combination according to claim 2 including Olesin 1 (AT4G25140), DGAT1 (AT2G19450), PDAT1 (AT5g13640) and WRI1 (AT3G54320).

17. The combination according to claim 16 further including a gene encoding a medium chain thioesterase protein.

18. The combination according to claim 16 further including genes encoding transcription factors selected from the group consisting of LEC1 and FUS2.

19. The combination according to claim 16 further including genes for inhibition of competing pathways.

20. The combination according to claim 19 wherein the competing pathway is the starch biosynthetic pathway.

21. The combination according to claim 20 further including the competing β-oxidation pathway.

22. A method for enhancing accumulation of triacylglycerol fatty acids in vegetative tissues of plants comprising introduction into a plant of a combination of genes for expression therein selected from the group consisting of genes which generally increase fatty acid synthesis, genes encoding oleosin or similar proteins, genes encoding diacylglycerol acyltransferases, genes encoding phospholipid: diacylglycerol acyltransferases, genes encoding medium chain thioesterases and combinations thereof.

23. The method according to claim 22 wherein the combination further includes genes encoding transcription factors and genes for inhibition of competing pathways.

24. An altered crop plant, derived from a parent plant, each plant having one or more vegetative tissues having a weight percent of triacylglycerol compounds (TAGS) wherein the weight percent of TAGs in said one or more vegetative tissues of said altered crop plant is at least two-fold higher than the weight percent of TAGs in the one or more vegetative tissues of said parent plant.

25. The altered crop plant of claim 24 wherein the weight percent of TAGs in the vegetative tissues is at least five-fold higher.

26. The altered crop plant of claim 25 wherein the weight percent of TAGs in the vegetative tissues is at least ten-fold higher.

27. The altered crop plant of claim 26 wherein the weight percent of TAGs in the vegetative tissues is at least twenty-fold higher.

28. An altered crop plant, derived from a parent plant, each plant having one or more vegetative tissues and said vegetative tissues having harvestable triacylglycerol compounds (TAGs) wherein the harvestable TAGs from said one or more vegetative tissues of said altered crop plant is twenty-fold higher than the harvestable TAGs from said one or more vegetative tissues of the parent plant.

29. The altered crop plant according to any one of claims 24 to 28 selected from the group consisting of corn, soybean, rice, canola, wheat, oat, sunflower, sorghum, sugar cane and camelina.

30. The TAG obtained from the one or more vegetative tissues of the altered crop plant of claim 24 or claim 28.
